(19) United States
(12) Patent Application Publication
(10) Pub. No.: US 2009/0216122 A1
(43) Pub. Date: Aug. 27, 2009

(54) Method and apparatus to control therapy of moving objects in living body

(76) Inventor: YOUSRY FARAGALLA, Lorton, VA (US)

Correspondence Address: YOUSRY FARAGALLA, P O BOX 1500, LORTON, VA, 22199, US

(21) Appl. No.: 12/035,521

(22) Filed: Feb. 22, 2008

(51) Int. Cl. A61B 8/00; A61N 7/00

(52) U.S. Cl. 600/439

(57) ABSTRACT

The invention relates to a method and apparatus for controlling the generation of therapeutic energy waves when aimed at a moving target object inside the living body where some energy waves can miss their target if the object moves outside the energy waves' focal zone. The apparatus comprises of a therapy controller that is designed to connect with energy waves' generators to control energy waves' generation and also to receive the object position data through connection with a real-time medical imaging system. The invention offers precise targeting of moving objects and minimizes wasted energy waves and undesired adversary effects of aiming at other healthy tissues. The energy waves' therapy is intended for objects within a living body. The object is in particular selected from a concretion, an organ, a tissue, a diseased part or a tumor, notably a malignant tumor.

### FIELD OF THE INVENTION

This invention relates to improving the targeting of energy wave therapy of moving objects inside a living body to increase efficiency and safety.

### BACKGROUND OF THE INVENTION

The current advances in non-invasive use of energy waves to treat disease inside the living body without the need of invasive surgery have become extremely useful in recent years. These energy waves when focused have been used to disintegrate kidney stones, ablate tumors or treat diseased or damaged tissues.

The present invention relates to a method and apparatus to make this type of treatment more precise when used with energy wave machines available in the market. A living body is not a 100% mechanically static target fixed in position. Consequently, objects inside the living body that we target for treatment are also moving. Anesthesia is used mainly to immobilize a living body. Nevertheless, with anesthesia, there still exist many other movements, especially those resulting from physiological functions, like respiration which moves most abdominal and thoracic organs and tissues, intestinal motility, pulsating blood vessels and pumping heart.

As an example, the shock wave lithotripsy, a type of energy wave, has provided in the past 20 years a good alternative for the treatment of renal stones by a non-invasive technique rather than a traditional invasive surgery. However, the efficiency for stone fragmentation, in different types of shock wave lithotripters, is approximately 60-70% stone-free rate. This could be partially attributed to the stone movement caused by patient's respiration during treatment, resulting in inaccurate targeting of the moving stone by the shock waves. Up to date, there is no lithotripter in the market with highly accurate stone targeting to resolve the issue of stone movement.

### SUMMARY OF THE INVENTION

The objective of the present invention is to provide a real-time, automatic control of energy wave generation so that the therapy targeted at a moving object inside a living body is precise and does not miss the moving target.

Other objectives, advantages, and novel features of the invention will become more apparent from the following detailed description when taken in conjunction with the accompanying drawings.

### BRIEF DESCRIPTION OF THE DRAWINGS

FIG. 1 shows the arrangement of the real-time imaging system, energy wave generator, its focal zone, the moving target located inside the focal zone and the present invention; therapy controller.

FIG. 2 shows the arrangement of the real-time imaging system, energy wave generator, its focal zone, the moving target located outside the focal zone and the present invention; therapy controller.

### DETAILED DESCRIPTION OF THE PRESENT INVENTION

An invention is described that provides a means for controlling the generation of energy waves to ensure that the said waves are only produced to hit their intended target. At any instance the target moves outside of the focal zone the controller will send orders to withhold the generation of said waves. As a result of the capabilities of the present invention, the accuracy and safety of energy wave therapy are effectively enhanced. In the following description, specific details are described in order to provide a thorough understanding of the present invention.

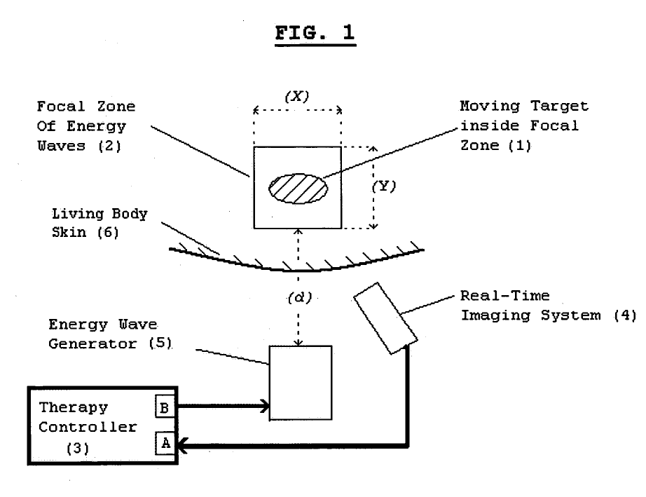

We proceed to the detailed description of the drawings to illustrate an overview of the application of the present invention in energy wave therapy. In FIG. 1 the energy wave generator (5) produces energy waves that are focused at a well defined focal zone (2) where the said waves are most effective in their intended therapy. The focal zone (2) as shown in the drawing and as visualized by the imaging system is defined by its transverse (X) and vertical (Y) dimensions. It is also defined by the distance (d) from its center to the source of the energy waves. Outside the focal zone, the energy waves are either not effective or have very minimal effect below the required therapy threshold. It is therefore the intention of any such therapy to maintain the therapy target (1) inside the focal zone of the energy waves (2). This can only be achieved by a real-time imaging system (4). In a living body (6) when the target (1) is located inside the focal zone (2) as visualized by real-time imaging system (4), the imaging system will send position information to the present invention; therapy controller (3) through its input connection (A). Then the Therapy controller will process the target (1) position with its pre-entered focal zone (2) position. As seen, in FIG. 1 the target lies inside focal zone. Consequently, the therapy controller will send orders through its output connection (B) to the energy wave generator (5) to produce energy waves.

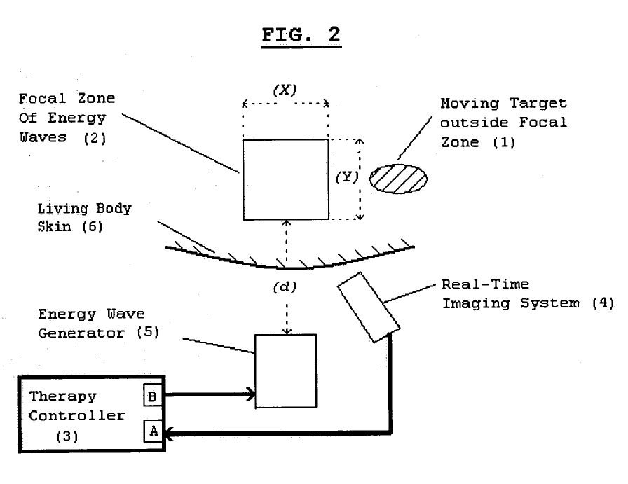

In FIG. 2, the target object (1) moves outside the focal zone (2) as visualized by the real-time imaging system (4) and relayed to the therapy controller (3) thru its input connection (A). Hence the therapy controller (3), through its connection (B) will order the energy wave generator to withhold any generation of energy waves. The withholding of energy waves generation will reverse if the target object returns to lie inside the focal zone. On the other hand, if the target continues to stay outside the focal zone and the generator (5) continues not to operate, this will alert the user to check the target position using the real-time imaging system (4) and to move the living body (6) in order to reposition the target (1) inside the focal zone (2). There are different types of real-time imaging systems used in conjunction with energy waves therapy, like ultrasound imaging systems and X-ray imaging systems. Some energy wave therapy systems offer a combination of both ultrasound and X-ray imaging. There are different energy waves that are used in therapy like shock waves, acoustic waves and high intensity focused ultrasound waves. The dimensions (X), (Y) and distance (d) of focal zone (2) differ according to the type of energy wave, type of apparatus, type of focusing and manufacturer's standards and specification. The present invention has the capability to reset the size and location of the focal zone stored in the therapy controller to match those of the specific energy wave generator used in therapy.

It is obvious to those skilled in the art that many modifications and or alterations may be made within the description of the energy wave generation. Additionally, any prior art energy wave generator and method of focusing may be controlled by the present invention.

We claim:

1) An apparatus for controlling the generation of energy waves used for therapy in a living body when said energy waves are aimed at a moving target compromising:
a) An input component receiving input from a real-time imaging system which monitors the said target and identifies its relation to the pre-defined focal zone of the energy waves,
b) A therapy controller processing the input data and
c) An output component sending orders to the energy waves generator to either generate the therapeutic energy wave when the target is inside focal zone or to withhold the energy wave generation when the target has moved outside the said focal zone.

2) An apparatus as in claim 1 wherein the source of energy waves may be more than one generator positioned geometrically to share a common focal zone.

3) An apparatus as in claim 1 wherein the real-time imaging system may be ultrasound imaging system.

4) An apparatus as in claim 1 wherein the real-time imaging system may be an X-ray imaging system.

5) An apparatus as in claim 1 wherein the real-time imaging system may be a combination of ultrasound and X-ray imaging systems.

6) An apparatus as in claim 1 wherein it can be adapted and connected to any model or type of energy waves generator.

7) An apparatus as in claim 1 wherein the type of energy waves are shock waves

8) An apparatus as in claim 2 wherein the type of energy waves are shock waves

9) An apparatus as in claim 3 wherein the type of energy waves are shock waves

10) An apparatus as in claim 4 wherein the type of energy waves are shock waves

11) An apparatus as in claim 1 wherein the type of energy waves are acoustic waves

12) An apparatus as in claim 1 wherein the type of energy waves are high intensity focused ultrasound waves

13) An apparatus as in claim 3 wherein the type of energy waves are high intensity focused ultrasound waves

14) An apparatus as in claim 1 wherein the pre-defined position of the focal zone of the energy waves can be relocated in the data processed by the therapy controller

15) An apparatus as in claim 1 wherein the pre-defined size and dimensions of the focal zone of the energy waves can be reset in the data processed by the therapy controller

16) A method for controlling the therapy of a living body using energy waves by monitoring the moving target and allowing the generation of the energy waves when the target exists inside the focal zone of said energy waves comprising: visualizing the target and coordinating its position with the pre-defined focal zone of the energy waves; sending orders to the energy wave generator or generators to either produce energy waves if the target is inside the focal zone or withhold energy waves generation if the target is outside the focal zone.
